(19) United States
(12) Patent Application Publication
(10) Pub. No.: US 2012/0031405 A1
(43) Pub. Date: Feb. 9, 2012

(54) METHODS AND SYSTEMS FOR CEREBRAL COOLING

(75) Inventors: Leroy D. Geist, Highlands Ranch, CO (US); Dennis J. Griffin, Englewood, CO (US)

(73) Assignee: CVA TECHNOLOGIES, LLC, Parker, CO (US)

(21) Appl. No.: 13/155,130

(22) Filed: Jun. 7, 2011

Related U.S. Application Data
(60) Provisional application No. 61/352,295, filed on Jun. 7, 2010.

(51) Int. Cl. A61M 16/10

(52) U.S. Cl. 128/204.15

(57) ABSTRACT

A brain cooling system includes a gas delivery system and a cooling apparatus. The gas delivery system may include an apparatus for establishing a desired pressure and flow rate for gases to be inhaled by a subject and an interface element. The pressurization element may comprise a continuous positive airway pressure (CPAP) device. The interface element may include a breathing mask, such as a nasal non-invasive ventilation (NIV) mask, or a nostril occlusive nasal delivery device. Such a brain cooling system may be used to treat cerebral hypoperfusion, as may occur with a cerebral vascular accident (CVA), such as a stroke, a traumatic brain injury, or cardiac arrest, or with conditions that may lead to a CVA or to cerebral hypoperfusion. In a cerebral hypoperfusion treatment method, cooled respiratory gases, which may include an elevated amount of oxygen, may be introduced, under an elevated air pressure that exceeds a normal, physiologic air pressure generated as a subject inhales spontaneously, into the nasal cavity of the subject.

### CROSS-REFERENCE TO RELATED APPLICATION

A claim for the benefit of priority under 35 U.S.C. §119(e) is hereby made to the Jun. 7, 2010, filing of U.S. Provisional Patent Application 61/352,295, titled “METHODS AND SYSTEMS FOR CEREBRAL COOLING,” the entire disclosure of which is, by this reference, hereby incorporated herein.

### TECHNICAL FIELD

The present invention relates generally to systems and methods for treating decreased blood flow to or through the brain, or cerebral hypoperfusion, which might occur in cerebral vascular accidents, such as stroke, cardiac arrest and traumatic brain injuries. In particular, the present invention relates to systems and methods that employ cooling techniques to stabilize a subject when cerebral hypoperfusion is suspected; for example, following a suspected cerebral vascular accident. Even more specifically, the present invention relates to methods in which cooled air or cooled, oxygen-rich gas is forced into the nasal cavity of a subject, and optionally into the subject's paranasal sinuses, to cool the subject's brain, as well as to systems that are configured to effect such a method. Some embodiments of the present invention, furthermore, include the simultaneous delivery of Continuous Positive Airway Pressure (CPAP) oxygen enriched, cooled gas to the lungs.

### SUMMARY

The present invention, in one aspect, includes methods for cooling, or lowering a temperature of, the brain of a subject. These methods may also be referred to as “cerebral cooling” methods. Brain cooling methods that incorporate teachings of the present invention may be useful for treating a decrease in the flow of blood through the brain, which is known in the art as “cerebral hypoperfusion.” Cerebral hypoperfusion may occur during a cerebral vascular accident, such as a stroke, a traumatic brain injury, or cardiac arrest, or with other conditions that may restrict or otherwise decrease the flow of blood into the brain.

In a various embodiments of a brain cooling method of the present invention, air, oxygen (O₂) or a gas mixture that includes elevated levels of oxygen (i.e., more than about 20.9, by molar content per volume) is cooled to a temperature below the normal body temperature (e.g., about 37° C., etc.) of a subject to whom the oxygen or gas mixture is to be administered. For the sake of simplicity, air, oxygen, and gas mixtures that include elevated levels of oxygen are also collectively referred to herein as “respiratory gas.” Respiratory gas that comprises substantially pure oxygen, as well as respiratory gas that includes above-normal amounts of oxygen (e.g., greater than about 20.9%, by molar content per volume, etc.), are also referred to herein as “oxygen-rich gas.” The respiratory gas may be administered under a positive pressure, which may exceed the normal, physiologic air pressure generated as the subject inhales spontaneously, on his or her own. In addition, the respiratory gas may be administered at a flow rate that exceeds the rate at which the subject normally inhales air. In some embodiments, the respiratory gas may be administered under continuous positive airway pressure. The manner in which cooled respiratory gas is delivered may provide control over and, thus, enable programming of, the rate at which a subject's brain (and body) are cooled.

The present invention also includes various embodiments of methods for returning the temperature of a subject's brain to a state of normal thermia (e.g., normal body temperature). These methods may also be referred to as “rewarming” methods. Rewarming may be effected at a rate that is programmed or otherwise controlled in such a way as to prevent a subject from entering into a state of shock.

Techniques of the present invention may also be used to control the core temperature of a subject's body. Cooling and/or rewarming of a subject's core temperature may also be effected at a controlled rate.

In another aspect, the present invention includes various embodiments of brain cooling systems. A brain cooling system of the present invention includes a gas delivery system and a cooling apparatus. The gas delivery system may include an oxygen source, an apparatus for establishing a desired pressure and flow rate for respiratory gas to be inhaled by a subject, and an interface element. The interface element receives respiratory gas from the pressurization element through an inspiratory breathing tube. In some embodiments, the pressurization element may comprise a continuous positive airway pressure (CPAP) device. The interface element, which receives respiratory gas from the pressurization element through an inspiratory breathing conduit, may include a breathing mask. Such a brain cooling system may be used to treat cerebral hypoperfusion, as may occur during a cerebral vascular accident (CVA), such as a stroke, traumatic brain injury, or cardiac arrest, or with any other condition that may inhibit or slow the flow of blood to a subject's brain and, therefore, may result in a CVA or, more broadly, in cerebral hypoperfusion.

Some embodiments of brain cooling systems that incorporate teachings of the present invention may also include temperature monitoring apparatus. The temperature monitoring apparatus may be configured to determine the temperature of a subject's brain, the subject's core temperature, or both the brain temperature and the core temperature of the subject.

A brain cooling system of the present invention may be configured to rewarm a subject's body and/or to maintain the subject's core temperature at or above a predetermined temperature. Warming may be effected by the cooling apparatus, or by a separate heating component.

In an additional aspect, the present invention includes a cooling apparatus. A cooling apparatus of the present invention, which may be used in conjunction with a brain cooling system, includes a conduit and a cooling element within the conduit. The cooling element may, in some embodiments, include a substrate that carries a channel through which a heat transfer fluid may flow. The elongate channel may travel a path that extends across various locations of an area of the substrate (e.g., a serpentine path, a meandering path, etc.). The path includes an inlet end, into which a cooled heat transfer fluid may flow, as well as an outlet end out of which a warmer heat transfer fluid may flow. The cooling element may be folded (e.g., in an accordion-like arrangement, rolled, etc.) to facilitate its placement within the conduit. When placed within the conduit in a folded configuration, the cooling element may define one or more narrow passages that extend along at least a portion of the length of the conduit in a way that enables fluid, such as respiratory gas, to flow through the conduit. Such a folded configuration of the cooling element may increase an internal surface area within a portion of the conduit. In addition to the conduit and the cooling element, a cooling apparatus of the present invention may include a fluid refrigeration apparatus for cooling the heat transfer fluid, as well as a cool fluid transfer conduit for providing cooled heat transfer fluid from the outlet of the refrigeration apparatus to the inlet end of the channel of the cooling element and a warm fluid transfer conduit for transporting warmer heat transfer fluid from the outlet end of the channel of the cooling element to the inlet of the refrigeration apparatus.

Other aspects and embodiments, as well as features and advantages of various aspects and embodiments, of the present invention will become apparent to those of skill in the art through consideration of the ensuring description, the accompanying drawings, and the appended claims.

### BRIEF DESCRIPTION OF THE DRAWINGS

In the drawings:

FIG. 1 is a schematic representation of an embodiment of a brain cooling system that incorporates teachings of the present invention;

FIG. 2 is a top view of an embodiment of a cooling apparatus that may be included in a system such as that shown in FIG. 1;

FIG. 3 is a cross-sectional view of the cooling apparatus of FIG. 2, taken along line 3-3 of FIG. 2; and

FIG. 4 is illustrates the embodiment of cooling apparatus of FIGS. 2 and 3 in a rolled configuration, disposed within a conduit; for example, the conduit of a brain cooling system such as that depicted by FIG. 1.

### DETAILED DESCRIPTION

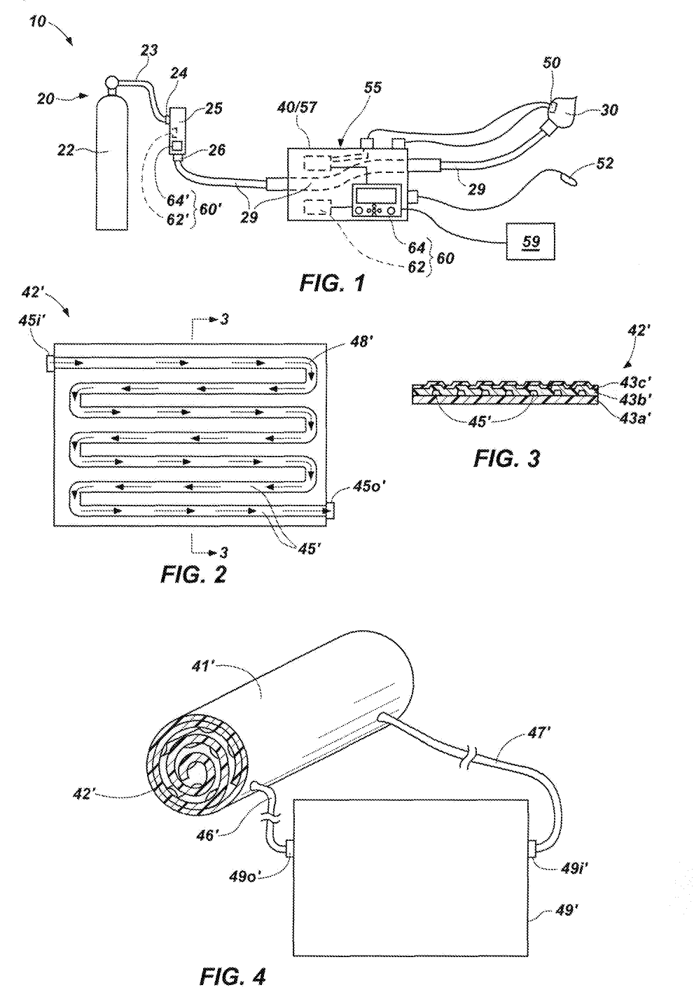

With reference to FIG. 1, an embodiment of a brain cooling system 10 is illustrated. Brain cooling system 10 includes a gas delivery system 20 and a cooling apparatus 40. The gas delivery system 20 of a brain cooling system may include a pressurization apparatus 25 and an interface element 30, which is configured to deliver cooled air or gas under positive pressure to the nasal cavity of a subject.

The pressurization apparatus 25 of a gas delivery system 20 of a brain cooling system 10 of the present invention may receive air, oxygen, or an oxygen-rich mixture of gases from a source 22. The source 22 may comprise a source of substantially pure oxygen (e.g., a gas tank, etc., containing the oxygen), the atmosphere, or a combination of the source of oxygen and the atmosphere. Such respiratory gas may be communicated from the source 22 to an inlet 24 of the pressurization apparatus 25 through a source conduit 23, as illustrated, or directly. In some embodiments, the pressurization apparatus 25 may also draw air from its surrounding environment. In such an embodiment, the air may be mixed with the oxygen or oxygen-rich mixture of gases from the source 22.

The pressurization apparatus 25 compresses the respiratory gas so that it may be delivered, under positive pressure, to an outlet 26 of the pressurization apparatus, and through an inspiratory breathing conduit 29 that communicates with the interface element 30. In some embodiments, the inspiratory breathing conduit 29 may be coupled directly to the outlet 26 of the pressurization apparatus 25.

More specifically, the pressurization apparatus 25 of a gas delivery system 20 of a brain cooling system 10 of the present invention may be configured to compress the respiratory gas to a pressure that exceeds the “normal” physiologic pressure of a subject's airway. The pressurization apparatus 25 may be configured to compress the respiratory gas to a pressure of about 0.5 kPa or more. In some embodiments, the pressurization apparatus 25 may compress the respiratory gas to a pressure of up to about 2 kPa.

Additionally, in some embodiments of a gas delivery system 20, the pressurization apparatus 25, its outlet 26, and the inspiratory breathing conduit 29 that communicates with the outlet 26 may be configured to deliver the respiratory gas at a flow rate of at least about 25 liters per minute (e.g., 30 liters per minute, 50 liters per minute, 60 liters per minute, etc.). In some embodiments, respiratory gas may be delivered at a rate of up to about 150 liters per minute.

Compression of the oxygen-rich gas may be effected in a substantially continuous manner, such that the pressurization apparatus 25 may deliver, through its outlet 26, respiratory gas at a substantially constant positive pressure. Without limiting the scope of the present invention, the phrase “substantially constant,” when used in conjunction with “positive pressure,” includes variations of about 10% or less from a predetermined positive pressure.

Moreover, the respiratory gas may be delivered by the pressurization apparatus 25 at a substantially constant rate of flow. “Substantially constant,” when used in reference to “rate of flow” or “flow rate,” includes, but is not limited to, flow rates that vary by no more than about 10% from a predetermined flow rate.

In a specific embodiment, the pressurization apparatus 25 comprises a continuous positive airway pressure (CPAP) apparatus. In such an embodiment, the interface element 30 may be configured to deliver a constant, uninterrupted (e.g., by exhalation, etc.) flow of cooled respiratory gas to the nasal cavity of a subject. The interface element 30 may comprise a nasal mask (e.g., a so-called “nasal non-invasive ventilation mask,” or “nasal NIV mask,” etc.), which is configured for placement over a portion of a subject's face. As an alternative, a so-called “nostril occlusive nasal delivery device” (e.g., nasal prongs, a cannula style bi-level positive airway pressure (BiPAP) mask, nasal pillows, etc.) are useful as interface elements 30 with CPAP apparatuses.

Alternatively, the pressurization apparatus 25 may be a transport ventilator. When used to deliver cooled respiratory gas to a subject, a transport ventilator may operate in a non-invasive positive pressure ventilation (NPPV) mode. Of course, other suitable devices may also serve as the pressurization apparatus 25 of a gas delivery system 20 that may be employed by a brain cooling system 10 of the present invention.

The cooling apparatus 40 of a brain cooling system 10 that incorporates teachings of the present invention is associated with the gas delivery system 20 in such a way as to cool respiratory gas before the respiratory gas is delivered to a subject. In some embodiments, the cooling apparatus 40 may be located upstream of the pressurization apparatus 25. In other embodiments, such as that depicted by FIG. 1, the cooling apparatus 40 may be located downstream from the pressurization apparatus 25, between the pressurization apparatus 25 and the interface element 30. The cooling apparatus 40, or the location at which the cooling apparatus 40 cools the respiratory gas to be inhaled by a subject, may even be located just upstream (e.g., within about 18 inches, within about 12 inches, etc.) from the interface element 30, or from a location at which respiratory gas enters the nasal cavity of the subject. Placement of the cooling apparatus 40 in such close proximity to the nasal cavity may minimize the transfer of heat into the respiratory gas as it travels between the location where it is cooled (e.g., at the cooling apparatus 40) and the subject's nasal cavity, which may increase, and even optimize, the efficiency of the brain cooling system 10.

The cooling apparatus 40 may be configured to cool the respiratory gas to a desired, or predetermined, temperature (e.g., about 1° C., up to about 35° C., about 10° C. to about 20° C., about 15° C., etc.) or to a temperature within a desired range of temperatures. In some embodiments, the cooling apparatus 40 may utilize known convective heat transfer methods. As an example, the cooling apparatus 40 may include a radiator, heat sink-type configuration that removes heat from the respiratory gas and transfers that heat to the external environment. In some embodiments, the cooling apparatus 40 may employ a coolant (e.g., FREON, etc.). In other embodiments, the cooling apparatus 40 may use one or more thermoelectric Peltier effect devices, such as those manufactured by Tellurex Corporation of Traverse City, Mich., to draw heat directly from a radiator/heat sink.

Another specific embodiment of cooling apparatus 40′ that may be used in a brain cooling system 10 of the present invention is illustrated by FIGS. 2-4. Cooling apparatus 40′ includes a conduit 41′ and a cooling element 42′ configured to be disposed within the conduit 41′. Cool and warm fluid transport conduits 46′ and 47′ enable circulation of a heat transfer fluid 48′ between the cooling element 42′ and the fluid refrigeration apparatus 49′.

The conduit 41′ may comprise part of the source conduit 23 or of the inspiratory breathing conduit 29 of the gas delivery system 20 (FIG. 1). Alternatively, the conduit 41′ may be configured to be coupled to the source conduit 23 or the inspiratory breathing conduit 29, either at an intermediate location or at an end of the source conduit 23 or the inspiratory breathing conduit 29.

A specific embodiment of the cooling element 42′, which is shown in FIGS. 2 and 3, includes a substrate 43′ that carries at least one channel 45′. The substrate 41′ may comprise a pair of laminated thin sheets or films 43a′, 43b′. Each thin sheet or film 43a′, 43b′ may have a sufficient area to be folded or rolled into an element that may be disposed within, and occupy a significant portion (e.g., at least about ¼, at least about ⅓, at least about ½, at least about %, up to about ¾, etc.) of a cross-sectional area of the conduit 41′, as illustrated by FIG. 4. Without limiting the scope of the present invention, each thin sheet or film 43a′, 43b′ of the substrate 43′ may be formed from a polymer, such as the biaxially-oriented polyethylene terephthalate (boPET) polyester film marketed by E.I. du Pont de Nemours and Company as MYLAR®.

One of the thin sheets or films 43b′ may define the at least one channel 45′. As a non-limiting example, each channel 45′ may be formed (e.g., thermally, etc.) into the thin sheet or film 43b′, which may then be secured (e.g., by an adhesive, by thermal bonding, etc.) adhered to the other thin sheet or film 43a′ to complete each channel 45′. Thermal conductivity of the substrate 41′ may be enhanced by providing a film 43c′ (e.g., laminating a preformed film 43c′, forming a film 43c′ by a vapor deposition process, forming a film 43c′ by sputtering, etc., as known in the art) comprising a more thermally conductive material (e.g., a metal, such as aluminum, etc.) to at least one of the thin sheets or films 43a′ or 43b′, such as over the protruding areas of thin sheet or film 43b′ that define each channel 45′.

The channel 45′ formed between thin sheets or films 43a′ and 43b′ includes an inlet end 45i′ and an opposite outlet end 45o′. The cool fluid transport conduit 46′ establishes communication between an outlet 49o′ of the fluid refrigeration apparatus 49′ and the inlet end 45i′ of the channel 45′, while the warm fluid transport conduit 47′ establishes communication between the outlet end 45o′ of the channel 45′ and an inlet 49i′ of the fluid refrigeration apparatus 49′.

The fluid refrigeration apparatus 49′ may comprise a thermoelectric liquid chiller, such as that available from Solid State Cooling Systems of Wappingers Falls, N.Y., as the OASIS 160, or any other refrigeration device that may cool a heat transfer fluid 48′ to a desired temperature (e.g., from about 1° C. to about 35° C., about 10° C. to about 20° C., about 15° C., etc.).

With returned reference to FIG. 1, the brain cooling system 10 may also include one or more temperature sensors 50, 52. A temperature sensor 50 may be associated with some feature of the brain cooling system 10 (e.g., the cooling apparatus 40, the interface element 30, etc.). Alternatively, a temperature sensor 52 may be configured for use in directly monitoring the subject's temperature (e.g., a so-called “physiologic tunnel,” such as a medial canthal area on a subject's face (i.e., near the medial corner of each of the subject's eyes), which provides a direct measure of brain temperature; eardrum, or tympanic membrane temperature; temperature within the nasal cavity, etc.).

A temperature sensor 50 associated with a feature of the brain cooling system 10 may be configured to provide a measure of the temperature of respiratory gas. Without limiting the scope of the present invention, a temperature sensor 50 may be positioned at or adjacent to a location of the interface unit 30 where respiratory gas exits the interface unit and/or enters the nasal cavity of a subject. Such an arrangement may enable monitoring of a temperature the respiratory gas at a point of contact with the subject.

Direct monitoring of a subject's temperature may comprise a non-invasive measurement of the subject's brain temperature, in which case the temperature sensor 52 may comprise an apparatus configured to sense temperature at a physiological tunnel (e.g., a terminal branch of the superior ophthalmic vein, etc.) that communicates the temperature of the subject's brain to a location at or near a surface of the subject's body. While a variety of apparatuses may be configured to obtain such a measurement and are, therefore, within the scope of the present invention, specific examples of such a temperature sensor 52 are provided by U.S. Pat. No. 7,187,960 to Abreu, the entire disclosure of which is, by this reference, hereby incorporated herein.

As another non-limiting example, devices that sense microwaves emitted by a subject's brain may be used to directly and non-invasively monitor the temperature of the subject's brain. Such a device passively senses microwaves, which are emitted from the brain with intensities that correspond to the temperature of the brain. An embodiment of such a device is described by Bass, W. T., et al., “Brain Temperature Measurement by Radiometric Thermometry in Normal Term Infants and Infants Treated with Moderate Systemic Hypothermia for Hypoxic-Ischemic Encephalopathy,” Pediatric Academic Soc./Asian Soc. For Ped. Res. Joint Mtg.—Denver, Colo., USA (2011) and by “Researchers develop device to measure brain temperature non-invasively,” EurekAlert!, http://www.eurekalert.org/pub_releases/2011-05/chot-rdd50211.php (May 2, 2011), the entire disclosures of both of which are hereby incorporated herein, in their entireties, by this reference.

The temperature sensor 52 of a brain cooling system 10 of the present invention may be configured to provide a measure of the core temperature of a subject's body. By way of non-limiting example, a temperature sensor 52 that is configured to monitor the temperature of a subject's tympanic membrane may provide an indication of the subject's core temperature.

Measurements of the temperature within a subject's nasal cavity may be obtained by use of a temperature sensor 52 that is configured to be disposed within the subject's nasal cavity and to contact a surface of the subject's nasal cavity.

Of course, a brain cooling system 10 of the present invention may include one or more temperature sensors 52 that are configured to monitor any of the brain temperature, core temperature, nasal cavity temperature, any other useful temperature or any combination of the foregoing.

It may be desirable to include a humidification component 55 in a brain cooling system 10. A humidification component 55 may be configured to introduce humidity (e.g., water vapor, a mist, etc.) into the nasal cavity of the subject. Humidification of tissues in the nasal cavity may be desirable to counteract the drying that may occur with the prolonged introduction of dry respiratory gas into the subject's nasal cavity. Humidification may also facilitate evaporative cooling of the tissues of the nasal cavity and, thus, expedite cooling of the brain.

The humidification component 55 may be configured to operate in accordance with a program, in response to feedback provided by other components of the brain cooling system 10 (e.g., a humidity monitor, which may be separate from or combined with a temperature monitor, etc.) or on demand. Moisture may be provided continuously or intermittently (e.g., in a pulsed manner; i.e., at a constant or substantially constant frequency; etc.) by the humidification component 55. In some embodiments, the humidification component 55 may be associated with (e.g., communicate humidity to, etc.) the gas delivery system 20 of the brain cooling system 10.

Continuing reference to FIG. 1, in addition to being configured to cool a subject's brain, brain cooling system 10 may include one or more warming elements, which are configured to warm parts of a subject's body. Without limitation, a brain cooling system 10 may be equipped to increase the temperature of a subject's brain. Alternatively, or in addition, a brain cooling system 10 may include one or more components for managing a subject's core temperature.

A rewarming element 57 may enable use of brain cooling system 10 to facilitate a post-cooling increasing the temperature of a subject's brain, or “rewarming.” In some embodiments, the rewarming element 57 may comprise an element configured to heat respiratory gas. Such a rewarming element 57 may communicate with the gas delivery system 20, which may introduce heated respiratory gas (relative to the temperature of the previously delivered cool respiratory gas and, in some embodiments, gradually increasing over time) into the subject's nasal cavity. In some embodiments, the cooling apparatus 40 may also serve as a rewarming element 57. By way of non-limiting example, in embodiments where the cooling apparatus 40 comprises one or more thermoelectric Peltier effect devices, reversal of electrical current through the cooling apparatus heats, rather than cools, the side of the cooling apparatus 40 against which respiratory gas flows. Although the rewarming element 57 is depicted in FIG. 1 as comprising at least a part of the same element as the cooling apparatus 40, the cooling apparatus 40 and the rewarming element 57 may comprise separate elements. In embodiments where respiratory gases may not be cooled and heated at the same location, the gas delivery system 20 of the brain cooling system 10 may enable tailoring of the temperature of respiratory gas by enabling the selective flow of respiratory gas through or past cooling elements and/or heating elements.

A body warming element 59 may enable management of subject's core body temperature while other components (e.g., any combination of the cooling element 40, any rewarming element 57 and/or the gas delivery system 20, etc.) of the brain cooling system 10 control the subject's brain temperature. In a specific embodiment, the body warming element 59 may be configured to prevent cooling of a subject's body to hypothermic levels (e.g., a temperature of less than 32° C., etc.) while cooling the subject's brain. A non-limiting example of a body warming element 59 includes a warming blanket or pad.

Some embodiments of a brain cooling system 10 may also include a control system 60, which may comprise a processing element 62, such as a computer processor and associated memory, a microcontroller, or the like. The processing element 62 may be programmed to control operation of at least one of the pressurization apparatus 25, the cooling apparatus 40, any humidification component 55, any rewarming element 57, any body warming element 59, and one or more other elements of the brain cooling system 10. By controlling operation of one or more of the pressurization apparatus 25, the cooling apparatus 40 and other components of the brain cooling system 10, the control system 60 may provide control (e.g., enable programming, etc.) over the rate and/or extent of cooling and/or heating.

In addition to the processing element 62, a control system 60 of a brain cooling system 10 of the present invention may also include an input/output element 64 of a known type. The input/output element 64 may communicate with the processing element 62 in a way that enables a user to control operation of one or more other elements of the brain cooling system 10, such as the pressurization apparatus 25, the cooling apparatus 40, and/or another element of the brain cooling system 10.

In the depicted embodiment, the control system 60 is part of the cooling apparatus 40. The input/output element 64 may enable a user to select a desired temperature to which the respiratory gas will be cooled, or even to which the brain will be cooled. When a user enters such a selection (e.g., a target temperature, etc.) into the input/output element 64, the input/output element 64 generates and transmits signals to the processing element 62, which then correspondingly increases or decreases a temperature of the cooling apparatus 40, causing the cooling apparatus 40 to operate in the manner desired by the user. Conversely, the processing element 62 may, in conjunction with one or more sensors or monitors of the brain cooling system, monitor one or more parameters (e.g., the temperature at a specific location of or adjacent to the cooling apparatus 40; the temperature monitored by a sensor 50, 52 associated with the interface element 30 or the subject; the humidity within the subject's nasal cavity; etc.) and, in some embodiments, based on the information, or feedback, provided by such monitoring, provide one or more alarms that enable an individual to manually adjust the delivery of respiratory gas or automatically control operation of one or more features of the cooling apparatus 40 in response to the monitored parameter or parameters (e.g., the temperature of respiratory gas; preventing hypothermia as brain cooling continues, etc.).

Alternatively, or in addition, the pressurization apparatus 25 may include or have associated therewith a control system 60′. In some embodiments, the control system 60′ associated with the pressurization apparatus 25 may include a processing element 62′ and an input/output element 64′. The input/output element 64′ may enable a user to select one or more of a desired gas mixture (e.g., a particular amount of oxygen), a desired pressure, and a desired flow rate of the respiratory gas to be delivered by the pressurization apparatus 25. Upon receiving a particular input command, the input/output element 64′ may generate corresponding signals, which are transmitted to the processing element 62′. Those signals are then processed by the processing element 62′, which may be programmed to generate and output signals that control operation of one or more features of the pressurization apparatus 25 to operate in the manner desired by the user. In some embodiments, the processing element 62′ may also receive signals from one or more sensors within the pressurization apparatus 25 or from one or more sensors associated with another component (e.g., the source conduit 23, the inspiratory breathing conduit 29, etc.) of the gas delivery system 20. A processing element 62′ that receives such signals may be programmed to automatically operate one or more features of the pressurization apparatus 25 in such a way that one or more desired parameters (e.g, gas mix, pressure, flow rate, etc.) are substantially constantly maintained by the pressurization apparatus 25.

Signals from one or more other sensors of the brain cooling system (e.g., temperature sensor 52, etc.) may be transmitted to and/or received by a processing element 62′ associated with the pressurization apparatus 25. The processing element 62′ may then automatically control operation of the pressurization apparatus 25 (e.g., increase or decrease the rate at which respiratory gases flow, etc.) in such a way as to provide a desired effect, such as a decrease or an increase in the temperature of the subject's brain, a change in the rate at which the subject's brain temperature increases or decreases or the like.

In other embodiments, a single control system may control operation of both the cooling apparatus 40 and the gas delivery system 20, as well as any humidification component 55, any rewarming element 57 and/or body warming element 59.

A method for cooling the brain of a subject in accordance with teachings of the present invention may be useful for treating a reduction in the flow of blood into and/or through the subject's brain, or cerebral hypoperfusion. Cerebral hypoperfusion may occur during a cerebral vascular accident, such as a stroke, a traumatic brain injury, or cardiac arrest, as well as in situations where a subject may be at risk for a cerebral vascular accident or cerebral hypoperfusion, such in patients experiencing congestive heart failure, pulmonary edema, or any other condition that may slow the flow of blood into the subject's brain. Such a brain cooling method includes delivering a respiratory gas that has been cooled to a temperature below the normal body temperature (e.g., about 37° C., etc.) of a subject, to the nasal cavity of the subject. By cooling the temperature within the subject's nasal cavity, the temperature of the subject's brain may also decrease. Introduction of the cool respiratory gas into the subject's nasal cavity may selectively lower the temperature of the subject's brain without inducing general hypothermia, a technique referred to as “selective brain cooling” (SBC). Some embodiments include reducing the temperature of the subject's nasal cavity to a temperature that enables the brain to be cooled at a desired rate while limiting counterproductive vasoconstriction (e.g., to a temperature of as low as about 15° C., etc.). As a non-limiting example, such cooling may be effected by initially delivering respiratory gas at a temperature of about 2° C. into the nasal cavity.

Decreasing a subject's brain temperature may also reduce the subject's body temperature. In some embodiments, brain cooling may be effected while minimizing any reduction in the subject's core body temperature. For example, for each one degree centigrade (1° C.) reduction in the temperature of a subject's brain, his or her core body temperature may only decrease by 0.2° C. (i.e., the ratio of change in brain temperature to the change in core temperature is high). In other embodiments, including those where brain temperature is reduced to provide for control over the rate at which the core temperature of a subject's body is decreased, the ratio of change in brain temperature to the change in core temperature may be lower.

In some embodiments, a method that incorporates teachings of the present invention may rapidly cool the subject's brain. Without limiting the scope of any aspect of the present invention, a brain cooling method of the present invention may be used to decrease the temperature of a subject's brain by about 2° C. or more within about 30 minutes. In a more specific embodiment, the temperature of a subject's brain may be cooled by about 3° C. or more within about 20 minutes, or even within about 15 minutes.

Once a desired brain temperature (e.g., a specific temperature, such as 32° C.; a temperature within a predefined range; etc.) has been achieved, that brain temperature may be maintained or substantially maintained (e.g., vary by less than about 10%, etc.). For example, after initially administering very cold (e.g., about 2° C., etc.) respiratory gas, the temperature of the respiratory gas may be gradually increased to about 15° C. to about 20° C.

The cooled respiratory gas that is administered to the subject may comprise air, or it may be oxygen-rich, meaning that the gas includes a higher than normal amount of oxygen, with “normal” being the roughly 20.9%, by molar content per volume, present in air. By delivering oxygen-rich gas, the rate at which oxygen flows into the subject's blood, and thus, into the subject's brain, may be increased.

Respiratory gas may be administered to the nasal cavity of a subject under a positive pressure, which may exceed the normal, physiologic air pressure generated as the subject inhales spontaneously, on his or her own. By administering respiratory gas under positive pressure, the alveoli within the lungs may expand fully or almost fully, which may increase, or even optimize, the transfer of carbon dioxide out of the subject's blood and oxygen into the subject's blood and, thus, into the subject's brain.

In addition, respiratory gas may be administered at a flow rate that exceeds the rate at which the subject normally inhales air. An increased flow rate may increase the rate at which carbon dioxide is flushed from the subject's lungs and replaced with fresh respiratory gas. Consequently, the rate at which oxygen is exchanged for carbon dioxide in the subject's blood and in the subject's brain may be increased. Non-limiting examples of the rate at which respiratory gas may be delivered to the subject's nasal cavity include rates of about 25 liters per minute and more (e.g., 30 liters per minute, 50 liters per minute, 60 liters per minute, etc.).

The pressure and/or flow rate at which respiratory gas is administered may be constant or substantially constant.

In a study involving pigs, respiratory gas having a temperature of 2° C. was introduced into the nasal cavity under continuous positive airway pressure at a flow rate of 60 liters per minute. Brain temperature, which was monitored directly, decreased by 2° C. in eight (8) minutes and by 3° C. in 17 minutes.

Any of the foregoing, alone or in combination, may increase, and even optimize, the amount of oxygen with the blood of a subject, and within the subject's brain. Increasing or optimizing the amount of oxygen supplied to a subject's brain may decrease or even minimize the likelihood of damage (i.e., ischemia) or further damage to the subject's brain.

Systems and methods of the present invention may also be used to control (e.g., lower, etc.) the core temperature of a subject's body. As with changes in the temperature of a subject's brain, the rate of change of a subject's core temperature may be controlled, or programmed.

Selective brain cooling and, thus, a brain cooling process and/or body cooling process according to the present invention may continue for a prolonged period of time. For example, brain cooling may continue for an extended period of time (e.g., about an hour; up to about 48 hours or longer; about 12 hours to about 24 hours; etc.).

After the brain and/or body of a subject has been cooled, particularly when the brain has been cooled for a prolonged period of time, it may be desirable to increase the temperature of the brain gradually, or in a controlled manner. Accordingly, the present invention also includes methods for rewarming the brain of a subject. The manner in which a subject's brain is rewarmed, including the rate at which rewarming is effected, may be controlled in such a way as to prevent any further damage to (i.e., ischemic activity in) the subject's brain. A caregiver may program the manner in which rewarming of the brain is effected. In some embodiments, the temperature of respiratory gas introduced into a subject's nasal cavity may be gradually increased from the most recent cool temperature to a temperature at or above the normal or desired temperature of the subject's brain.

In specific embodiments, rewarming may be effected by introducing respiratory gas having a temperature of about 33° C. to about 39° C. into a subject's nasal cavity. Even more specifically, cooler (e.g., about 33° C. to about 35° C., etc.) respiratory gas may initially be introduced into the subject's nasal cavity, then the temperature of the respiratory gas may be gradually increased (e.g., to a temperature of about 37° C. to about 39° C., etc.). Rewarming may continue until the temperature of the subject's brain returns to normal (e.g., about 37° C.).

Although the foregoing description contains many specifics, these should not be construed as limiting the scope of any of the appended claims, but merely as providing information pertinent to some specific embodiments that may fall within the scopes of the appended claims. Other embodiments may also be devised which lie within the scopes of the appended claims. Features from different embodiments may be employed in combination. The scopes of the appended include all legal equivalents. All additions, deletions and modifications to the invention, as disclosed herein, that fall within the meaning and scopes of the claims are to be embraced thereby.

What is claimed:

1. A method for treating cerebral hypoperfusion, comprising:
cooling respiratory gas to a temperature below body temperature to provide cooled respiratory gas; and
introducing the respiratory gas into at least a nasal cavity of a subject under an elevated air pressure that exceeds a normal, physiologic air pressure generated as the subject inhales spontaneously.

2. The method of claim 1, wherein introducing the respiratory gas comprises introducing the respiratory gas at a flow rate that exceeds a normal physiologic flow rate when the subject inhales spontaneously.

3. The method of claim 1, wherein cooling the respiratory gas comprises cooling the respiratory gas to a temperature of about 35° C. or less.

4. The method of claim 3, wherein cooling the respiratory gas comprises cooling the respiratory gas to a temperature in a range of about 1° C. to about 35° C.

5. The method of claim 4, wherein cooling the respiratory gas comprises cooling the respiratory gas to a temperature in a range of about 33° C. to about 35° C.

6. The method of claim 1, wherein cooling the respiratory gas comprises initially cooling the respiratory gas to a temperature of about 2° C. and subsequently increasing the temperature of the respiratory gas to about 15° C. to about 20° C.

7. The method of claim 1, further comprising:
directly monitoring a temperature of the brain.

8. The method of claim 7, wherein directly monitoring the temperature of the brain comprises non-invasively monitoring the temperature of the brain.

9. The method of claim 7, further comprising:
changing a temperature to which the respiratory gas is cooled to substantially maintain the temperature of the brain at a minimum threshold temperature.

10. The method of claim 7, wherein introducing decreases the temperature of the brain to a minimum threshold temperature of about 32° C. to about 35° C.

11. The method of claim 1, wherein introducing is controlled to substantially maintain the temperature of the brain within a range of about 32° C. to about 35° C.

12. The method of claim 1, wherein cooling comprises cooling a gas mixture including an elevated level of oxygen.

13. The method of claim 12, wherein cooling the gas mixture comprises cooling a gas mixture including at least about 28% oxygen.

14. The method of claim 13, wherein cooling the gas mixture comprises cooling a gas mixture including about 28% oxygen to about 50% oxygen.

15. The method of claim 1, wherein introducing comprises introducing the cooled oxygen or the cooled gas mixture into at least the nasal cavity of the subject at a continuous positive airway pressure.

16. The method of claim 1, wherein introducing comprises introducing the cooled oxygen or the cooled gas mixture into at least the nasal cavity of the subject under a pressure of at least about 0.5 kPa.

17. The method of claim 16, wherein introducing comprises introducing the cooled respiratory gas into at least the nasal cavity of the subject under a pressure of about 0.5 kPa to about 2 kPa.

18. The method of claim 1, wherein introducing comprises introducing the cooled respiratory gas into at least the nasal cavity of the subject at a flow rate of at least about 25 liters per minute.

19. The method of claim 1, wherein introducing the respiratory gas comprises introducing the respiratory gas at a flow rate of at least 30 liters per minute.

20. The method of claim 1, wherein introducing the respiratory gas comprises introducing the respiratory gas at a flow rate of at least about 60 liters per minute.

21. The method of claim 1, wherein the acts of cooling and introducing decrease a temperature of a brain of the subject by at least about 2° C. within at most about 30 minutes.

22. The method of claim 21, wherein the acts of cooling and introducing decrease the temperature of the brain of the subject by at least about 3° C. within at most about 20 minutes.

23. The method of claim 1, further comprising:
substantially maintaining a temperature of a brain of the subject at a depressed temperature.

24. The method of claim 23, further comprising:
after substantially maintaining the temperature of the brain of the subject at the depressed temperature, rewarming the brain.

25. The method of claim 24, wherein rewarming the brain includes introducing respiratory gas at a temperature of about 33° C. to about 39° into the nasal cavity of the subject.

26. The method of claim 24, wherein rewarming the brain includes gradually increasing the temperature of the respiratory gas while continually introducing the respiratory gas into the nasal cavity of the subject.

27. The method of claim 1, wherein cooling comprises maintaining the nasal cavity of the subject at a temperature that limits vasoconstriction.

28. The method of claim 27, wherein cooling comprises maintaining the nasal cavity of the subject at a temperature of at least about 15° C.

29. The method of claim 1, further comprising:
introducing humidity into the nasal cavity of the subject.

30. The method of claim 29, wherein introducing the humidity comprises intermittently introducing humidity into the nasal cavity.

31. A system for cooling a brain of a subject, comprising:
a gas delivery system for pressurizing respiratory gas and providing the respiratory gas to a nasal cavity of a subject at an elevated air pressure that exceeds a normal, physiologic air pressure generated as a subject inhales spontaneously; and
a cooling apparatus in communication with the gas delivery system for cooling the respiratory gas.

32. The system of claim 31, wherein the gas delivery system includes:
a source of respiratory gas;
a flow generator for receiving respiratory gas from the source and expelling the respiratory gas at a predetermined pressure and about a predetermined flow rate; and
an interface element for receiving the respiratory gas from the flow generator and delivering the respiratory gas to the nasal cavity of the subject.

33. The system of claim 32, wherein the flow generator receives substantially pure oxygen.

34. The system of claim 32, wherein the flow generator comprises a continuous positive airway pressure (CPAP) apparatus.

35. The system of claim 34, wherein the interface element comprises a nasal non-invasive ventilation mask.

36. The system of claim 31, wherein the cooling apparatus includes a cooling element located along a conduit between the flow generator and the interface element.

37. The system of claim 31, further comprising:
a temperature probe.

38. The system of claim 37, wherein the temperature probe is associated with the interface element.

39. The system of claim 38, wherein the temperature probe is configured to monitor a temperature of respiratory gas as the respiratory gas is delivered to the nasal cavity of the subject.

40. The system of claim 37, wherein the temperature probe is configured to obtain a direct brain temperature measurement.

41. The system of claim 40, further comprising:
a control element in communication with the temperature probe and configured to control operation of at least one of the cooling apparatus and the gas delivery system responsive to signals received from the temperature probe.

42. The system of claim 41, further comprising:
another temperature probe configured to monitor a core temperature of the subject.

43. The system of claim 41, wherein the another temperature probe communicates with the control element.

44. The system of claim 43, wherein the control element controls operation of at least one of the cooling apparatus and the gas delivery system in response to signals received form the another temperature probe.

45. The system of claim 43, further comprising:
a rewarming element operable under control of the control element.

46. The system of claim 45, wherein the control element operates under control of user programming to warm the brain of the subject at a predetermined rate.

47. The system of claim 31, further comprising:
a humidity monitor.

48. The system of claim 47, further comprising:
a humidification component.

49. The system of claim 48, further comprising:
a control element in communication with the humidity monitor and configured to control operation of the humidification component in response to signals received from the humidity monitor.

50. The system of claim 37, further comprising:
a controller in communication with the temperature probe and the cooling element, the temperature probe being configured to generate signals, the controller being programmed to receive the signals, process the signals, and control operation of at least one of the cooling apparatus and the gas delivery system to achieve and maintain a predetermined temperature at the temperature probe.

51. The system of claim 31, wherein the cooling apparatus cools the respiratory gas at a location within about 18 inches from an entrance into the nasal cavity of the subject.

52. The system of claim 51, wherein the cooling apparatus cools the respiratory gas at a location within about 12 inches from an entrance into the nasal cavity of the subject.

53. A cooling apparatus, comprising:
a primary conduit;
a cooling element disposed within the conduit in a folded configuration providing at least a portion of a length of the primary conduit with an increased internal surface area in a manner that enables fluid to flow through the length of the conduit, the cooling element including:
a substrate;
a channel defined at least partially by the substrate and including an inlet end and an outlet end; and
a heat transfer fluid within the meandering channel;
a fluid refrigeration apparatus including an inlet and an outlet;
a cool fluid transport conduit establishing communication between the outlet of the fluid refrigeration apparatus and the inlet end of the channel of the cooling element; and
a warm fluid transport conduit establishing communication between the outlet end of the channel of the cooling element and the inlet of the fluid refrigeration apparatus.

54. The cooling apparatus of claim 53, wherein the folded configuration comprises a rolled configuration.

55. The cooling apparatus of claim 53, wherein the primary conduit is configured to be coupled along a length of an inspiratory breathing tube.

56. The cooling apparatus of claim 53, wherein the cooling element includes a meandering channel.

57. The cooling apparatus of claim 53, wherein the substrate of the cooling element comprises a flexible substrate.
